(19) United States
(12) Patent Application Publication
(10) Pub. No.: US 2021/0145089 A1
(43) Pub. Date: May 20, 2021

(54) Face mask frame with face care and blood circulation attachments

(71) Applicant: Eman Mahmoud Hussien Hassan, Redmond, WA (US)

(72) Inventor: Eman Mahmoud Hussien Hassan, Redmond, WA (US)

(21) Appl. No.: 17/120,312

(22) Filed: Dec. 14, 2020

(51) Int. Cl. A41D 13/11; A61N 5/06

(52) U.S. Cl. CPC A41D 13/1169; A61N 5/0616; A61F 13/00063; A61N 2005/0651; A61N 2005/0647

(57) ABSTRACT

The invention relates to introducing a bendable frame for viral protection and surgical face masks to help protect, soothe and circulate blood of irritated skin affected from wearing face masks for a long time specially during the time of a pandemic such as COVID-19, introducing flexible sticky tapes which help in moisturizing and massaging skin that is in contact with the face mask and also contains attachment socket for adding helpful attachments for the area under the face mask such small fan attachment to help dry skin from sweat without having to take off the face mask, light therapy attachment to help prevent acne or irritation and extra sockets for any further customized attachments.

### FIELD OF THE INVENTION

The field of the inventions is related to a multi feature face mask frame that gets attached to face masks including viral protection and surgical masks. This attachment helps protect and soothe people's face from irritation, bruising and help circulating blood under the mask along with cooling and drying the affected skin especially for people who use it for continued long hours per day.

### BACKGROUND OF THE INVENTION

Face masks have been widely and intensively used by healthcare workers, patients and all people around the world specially during a pandemic like COVID-19.

It has become very common that a person can wear a face mask for long hours each day especially healthcare workers who almost wear it 24/7.

During COVID-19 pandemic it has become obvious that although face masks can protect and save people's lives, there is a downside to them that they cause irritation of skin and causes the face to bruise and get hurt around the edges of the face mask, it also increases humidity and sweat under the mask, It has also been found that some sensitive skin gets acne and irritation around the chin because of lack of light exposure during the long hours of wearing the mask.

CBS News reported on Mar. 31, 2020 an article with a title “Exhausted doctors and nurses post images of their bruised faces after long shifts wearing protective gear” quoting that “Health care professionals on the front lines of the coronavirus battle are struggling against forces that are out of their control. Overcrowded hospitals, a lack of ventilators, the risk of getting infected themselves and the need for personal protective equipment (PPE) are just a few challenges these workers are facing.”. So, this shows how much wearing face masks for a long time can damage the face not just some bruises but also the effect of less blood circulation for the face in a consistent daily manner is a problem that needs to be addressed with all means possible.

During COVID-19 the previously stated news have become very common with the whole population not just healthcare workers, it has become necessary to wear the face masks for long hours while working, for example: grocery stores' workers, mail delivery services and all the workplaces that didn't closedown even during the pandemic to serve people's needs. And according to CDC regulations on the effective use of face masks is to wear them in a way that they fit snuggly against the size of your face and don't have gaps, this means that it will cause pressure and friction with the skin which are mostly the causes of getting skin problems in the long term.

Another concerning problem showed up during the summer when the heat started to increase and sweating under the face mask has become very common and intensive while it is not recommended to remove the mask every few minutes to dry off the sweat which could lead to more exposure to getting infected while doing that. The sweating problem can also cause the face mask itself to get wet very fast which makes it less or even not at all effective in infection prevention.

The invention of the face mask frame is targeting mainly the problem of the face suffering below the mask, so it helps soothe the skin and cool it down and dry the sweat under the skin without having people to remove the mask frequently and expose to infection.

### BRIEF SUMMARY OF THE INVENTION

The face mask frame is customizable bendable frame that acts as a frame to any type of viral protection or surgical face masks or any mask that has flexible material that have thin edges to attach to the frame. The frame has disposable attachments including tapes that can stick to the frame and sticks to the face instead of the face mask. These tapes contain soothing moisturizer gel that helps cool down the skin and helps in collagen generation and increase the blood circulation of the skin. Another type of the tapes are rechargeable massager tapes that get attached to the frame and vibrates and massages the skin to help relief the skin and the face muscles under the mask. the frame has customizable options to facilitate how people wear it according to their own preference including attaching ear loops or head loops or silicon tape.

The area of the frame that is under the face mask attached contains a couple of removable and replaceable attachments containing small rechargeable fan attachments to dry off sweat and decrease the heat under the mask, small rechargeable LED light therapy attachment to expose the skin to therapeutic light to decrease the potential of developing acne or inflammation and extra attachment sockets for any newly third party accessories to use and attach to face mask for example mic voice amplifier to help make the voice more clear and better heard while using the face mask.

### BRIEF DESCRIPTION OF DRAWINGS

FIG. 1 An illustration of the overall look of the face mask frame with all the attachments.

FIG. 2A Shows the clip in the frame that holds the face mask while it is open.

FIG. 2B Shows the clip in the frame that holds the face mask while it is closed over the mask to hold to mask tightly.

FIG. 3 Shows an example of a moisturizer tape with a peel-off side to stick to the face mask frame's side that is towards the skin.

FIG. 4 Shows the massager tape with a peel-off side to attach to the face mask frame and the other side touching the face's skin to massage the area under the frame every minute or customizable time.

FIG. 5 Illustrates the attachments socket in the face mask frame's interior edge.

FIG. 6 Illustrates the look of a small chargeable or battery fan attachment to be added inside one of the attachment sockets.

FIG. 7 Illustrates chargeable or battery light therapy attachment.

FIG. 8 Illustrates the optional silicon tape containing silicon around the top and bottom edges to stick the said frame to the face.

### DETAILED DESCRIPTION OF THE FACE MASK FRAME

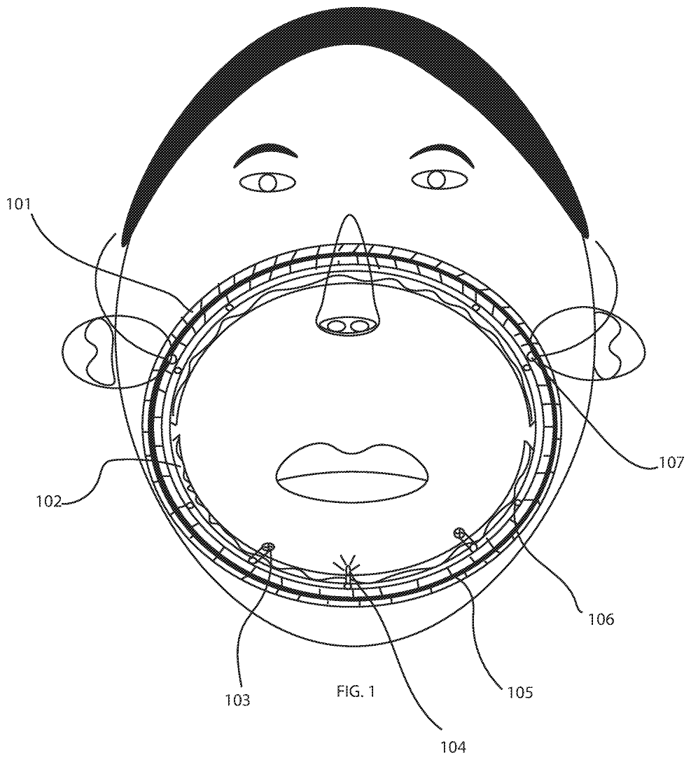

FIG. 1 shows overall picture of how the mask frame would look like, containing the following: mask frame 101, face mask clip 105, head/ear loops attachment clip 107, face side soothing and messaging attachable tapes 102, attachment sockets 106, removable fans attachment 103, removable LED light therapy attachment 104, open attachments sockets for extra custom accessories as one shown in 106.

The mask frame 101 could be made of woven cloth surrounding a strong kind of bendable metal strip so that it could be easily reshaped and attached to the face according to the user preference and the shape of the face mask that needs to be clipped to the said frame.

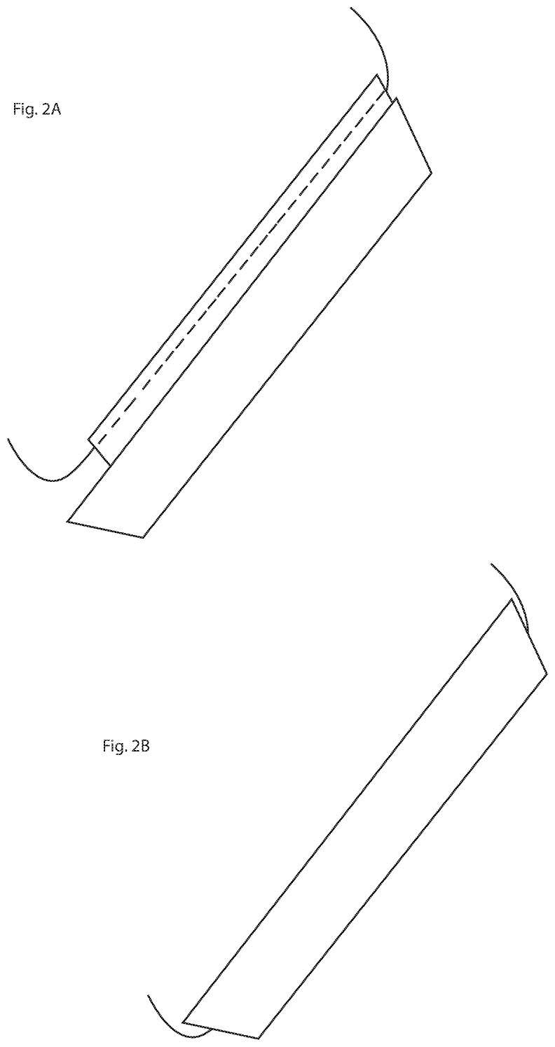

Face mask clip 105 is bendable plastic clip that holds the face mask edges to keep the mask snuggled completely around the frame as shown in FIG. 2A for the opened view and FIG. 2B for the closed view.

Two small-rounded plastic clips 107 attached to each side of the frame to attach either ear loop to hold the frame or head loop.

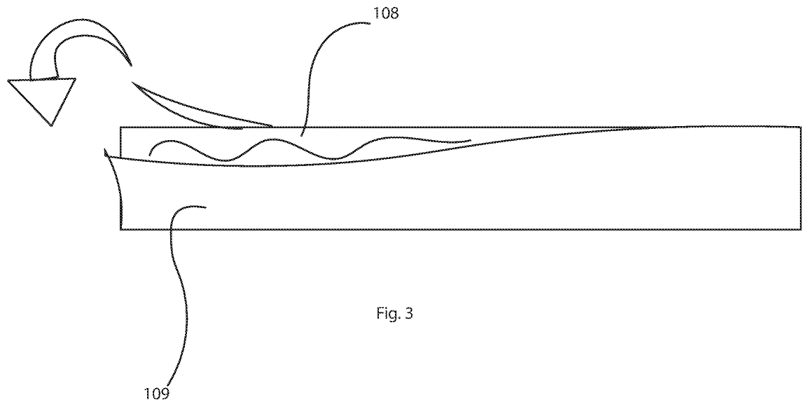

Face side soothing and messaging attachable tapes 102 as shown in detail in FIG. 3 which shows the soothing tape which is disposable. One side contains the soothing gel 108 with an easy peel off to protect it before use 109 and the gel 108 could also be manufactured with variety of moisturizers, pain reliefers or cooling materials to help cure and relief the skin while using the face mask for long hours, the other side of the tape contains sticky glue to attach to the cloth area designated for the tape at the skin facing side of the frame.

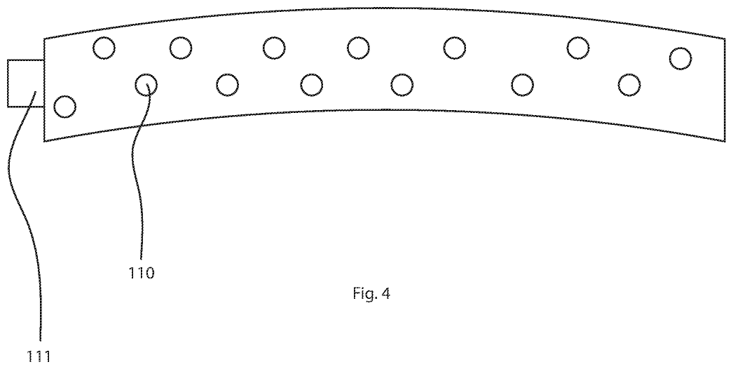

FIG. 4 shows the massaging tape which is made of washable woven cloth with disposable silicon layer surrounding vibrating massaging poles 110 that are rechargeable through a charging tip at one end of the tape 111. These poles vibrate the skin and slightly massage the area under the face mask frame that turns on every 10 minutes and runs for 30 seconds noting that timing and customization could be handled with multiple versions of the massaging tape.

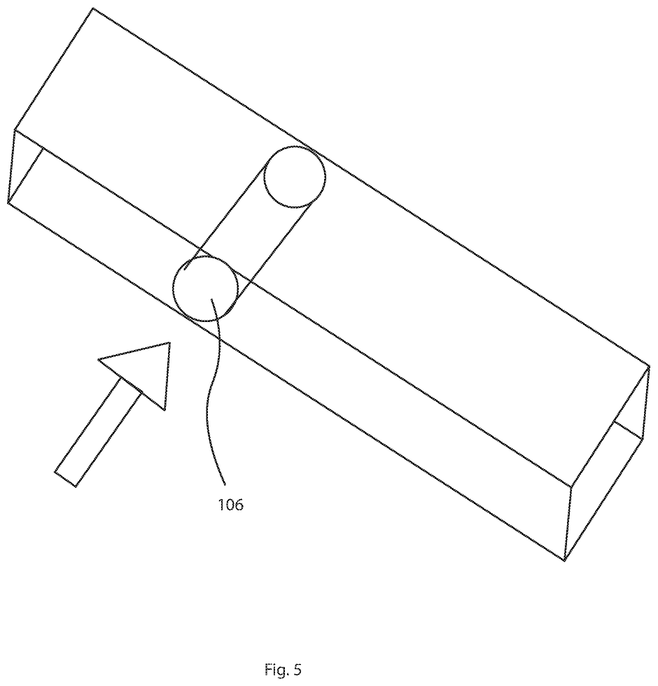

FIG. 5 shows attachment sockets 106 which are made of plastic that is inside the face mask frame cloth and they are open from the inside liner to have the attachments get hooked inside those sockets before clipping on the face mask to the frame to cover the intended area of the face.

The attachment parts that will get inside the attachment sockets are all having standard ending of plastic that is fitting inside the sockets while the other ending side could differ according to the type of the attachment and the feature it offers inside the mask under the condition that it doesn't irritate the face in any way.

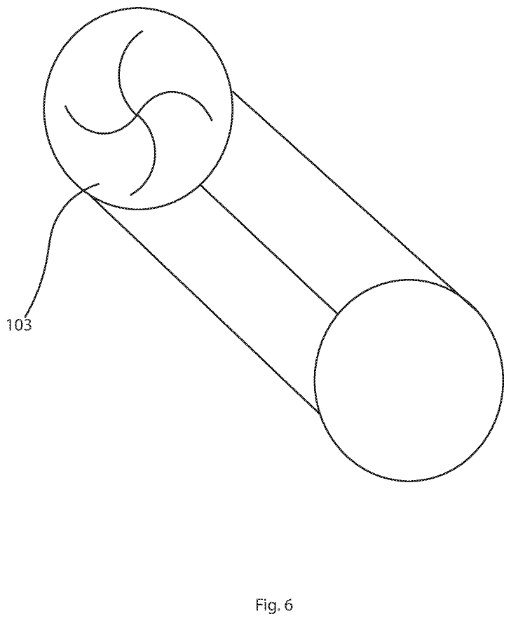

FIG. 6 shows removable fan attachment 103 that is a small micro fan that gets attached in the bottom sides of the frame directed to the top of the face mask to help cool down skin and decrease the heat under the face mask and help dry off any sweat on the skin without having to take off the face mask or touching the face with contaminated hands.

The fan could be made of plastic material with micro motor that could be chargeable or designed with battery.

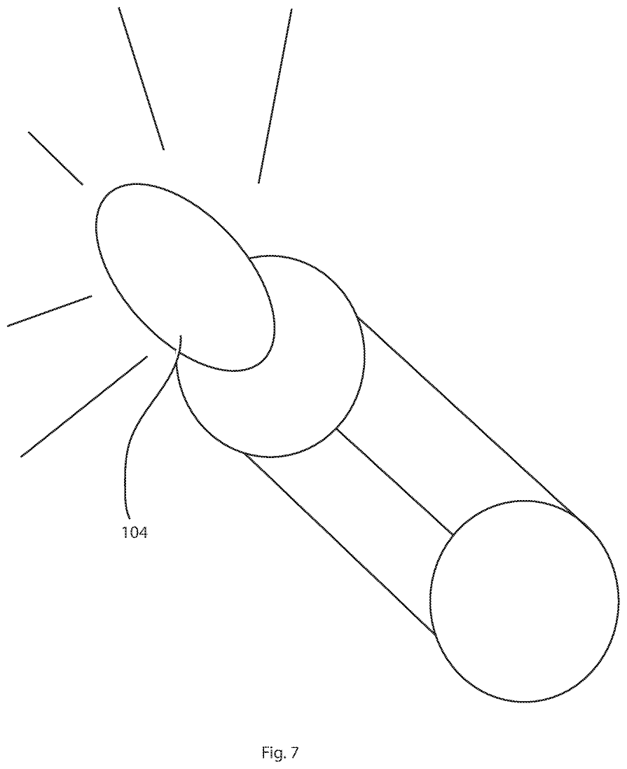

FIG. 7 shows light therapy attachment 104, light therapy is well known for preventing skin inflammation and acne which are also common side effects of using face masks, removable LED light attachment could be added in the middle of the chin area where it usually has most acne issues and could also be attached to any of the extra custom attachment sockets.

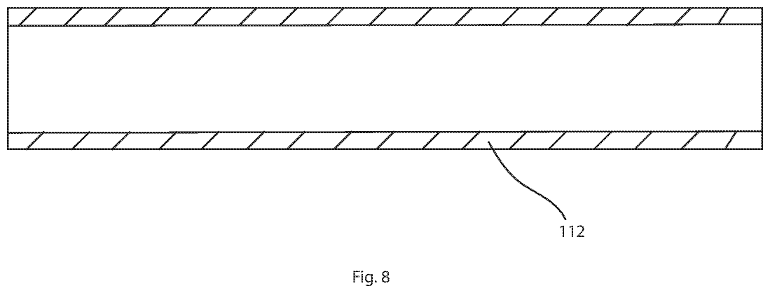

FIG. 8 shows attachable silicon tape are for having the option to stick the mask frame to the face without having to use ear or head loops, the tape is rectangular shape cloth with silicon the top and bottom edges 112 while the area in between would be used to attach the cooler or massager tapes shown in FIGS. 2A, 2B and 3.

Since there might be more needs in the future for features within the mask to be added as attachments, extra sockets are installed to the frame to allow for more attachments for example: voice amplifier attachment to help better hear the person wearing the mask clearly and loud enough so that they don't have to remove the mask in any situation that could expose them to infection.

What is claimed is:

1- A face mask frame comprising:
A bendable material to fit any shape of viral protection or surgical face masks to get attached to the said frame through an attachment clip; flexible ear and head loop clip on the sides of the said face mask frame to attach either ear or head loops to hold the said frame with the attached face mask; attachable sticky massaging and moisturizing tapes that stick to the side of the said frame facing the skin; attachment sockets for skin and face protection features for installing any customized face soothing or protection attachments; fans attachments to be installed in the said attachment sockets to help soothe or dry out sweat on the skin area under the face masks being attached to the said frame; LED light therapy attachments to be installed in the attachment sockets to give light therapy to the said skin area under the face masks being attached to the said frame.

2- The invention of claim 1 wherein the method of manufacturing an attaching tool to attach the face mask to a frame.

3- The invention of claim 1 wherein silicon tape attachments are manufactured to be attached to the skin facing side of the frame to stick the said frame to the face.

4- The invention of claim 1 wherein attachment sockets are within the frame structure or inside the frame material.

5- The invention of claim 1 where custom new attachments to the said frame is allowed.

6- The invention of claim 1 wherein a massager tape containing vibrating massaging poles with removable silicon tape on top to stick to the skin.

7- The method of face mask tape attachments having soothing gel, moisturizers or any skin curing material.

8- The method of massaging skin under face masks.
